(19) United States
(12) Patent Application Publication
(10) Pub. No.: US 2019/0313679 A1
(43) Pub. Date: Oct. 17, 2019

(54) SUGAR-DIPEPTIDE CONJUGATES AS FLAVOR MOLECULES

(71) Applicant: NESTEC S.A., Vevey (CH)

(72) Inventors: Candice Marie Smarrito-Menozzi, Belmont-sur-Lausanne (CH); Florian Viton, Lausanne 26 (CH); Maria Eugenia Barcos, Epalinges (CH); Sonia Manganiello, Neuchatel (CH)

(21) Appl. No.: 16/314,726

(22) PCT Filed: Jul. 6, 2017
(86) PCT No.: PCT/EP2017/066876

§ 371 Date: Jan. 2, 2019

(30) Foreign Application Priority Data
Jul. 7, 2016 (EP) 16178414.5

(51) Int. Cl. A23L 27/20; C07H 15/12; C07K 5/072; A23L 23/10; A23L 27/21; A23L 27/00

(52) U.S. Cl. CPC A23L 27/2052; C07H 15/12; C07K 5/06104; A23V 2002/00; A23L 27/21; A23L 27/86; A23L 27/88; A23L 23/10

(57) ABSTRACT

The present invention relates to compounds of formula (I) and compositions comprising them for use in enhancing umami taste and/or saltiness of a food product.

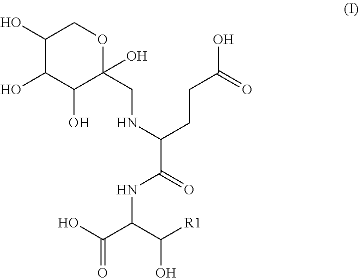

The present invention relates to compounds and compositions for use in enhancing umami taste and/or saltiness of food products.

Many foods that are consumed today are rich in umami and/or meaty taste and flavor. Umami or meaty taste of a food product can for example be achieved or enhanced by adding separately monosodium glutamate (MSG) and/or the ribonucleotides GMP and IMP into those culinary recipes. Many such taste enhancers are available today and are used for various different culinary applications and in various different forms such as pastes, powders, liquids, compressed cubes or granules.

The addition of culinary additives helps to provide deliciousness and to enhance taste and flavor properties of food products. And indeed, all around the world taste and flavor is perceived as one of the key attributes of a high quality meal. Hence, a lot of research efforts goes into the identification and analysis of new molecules providing deliciousness, and enhanced taste and flavor properties of foods.

Also common kitchen salt, basically sodium chloride, plays an important role in influencing and enhancing the taste and flavor of food products. And salt also by itself is an important taste component. It is established today, that the sensation of taste of a food product is composed of five basic tastes, i.e. sweetness, sourness, saltiness, bitterness and umami. Those different tastes are captured on our tongue by specifically differentiated taste buds. Thereby, bitter and sour foods are usually found rather unpleasant, while sweet, salty and umami tasting food products are generally regarded as providing a pleasurable sensation upon eating such food products.

Although it is well recognized that consumption of a certain amount of salt is indispensable for a healthy human life, the tendency of today's consumption and diets is that too much salt, particularly sodium chloride, is consumed on an individual basis and worldwide. It is recognized today that ingesting excessive quantities of sodium salt raises the risk of hypertension, kidney diseases and heart diseases. Hence, there is still a need in the art to provide new flavorings which allow the reduction of sodium salts in nutritional diets, and which still can provide the taste enhancing effect and saltiness as for example traditional kitchen salt.

M. Tamura et al., Agric. Biol. Chem., 1989, 53 (2), 319-325, described the relationship between taste and primary structure of a delicious peptide from beef soup that produced a salty and umami taste as well as delicious taste. They referred to earlier work where they for example synthesized ornithinyl-taurine and found that it produced a salty taste. Similar taste effect was discovered with similar other dipeptides.

EP2253227A1 discloses a series of glutamic acid containing dipeptide molecules which act as salty taste enhancers when added to an enzymatic decomposition product of a protein material or a basic amino acid, especially arginine. Such salty taste enhancers can then compensate for insufficient salty taste when attempting to reduce the salt content of a food product.

The object of the present invention is to improve the state of the art and to provide an alternative or improved solution to the prior art to overcome at least some of the inconveniences described above. Particularly, the object of the present invention is to provide an alternative or improved solution for enhancing the taste of food products. Particularly, the object of the present invention is to improve the taste, as for example the delicious, umami and/or salty taste, of a food product. The object or the present invention is also to provide a solution for compensating for the lost saltiness when lowering the effective amount of sodium salt in a food product.

The object of the present invention is achieved by the subject matter of the independent claims. The dependent claims further develop the idea of the present invention.

Accordingly, the present invention provides in a first aspect a compound of the general formula I,

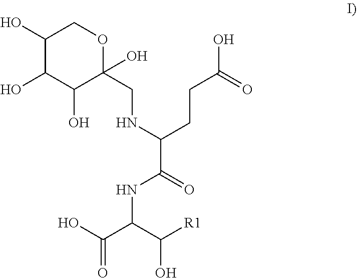

wherein R1 is hydrogen (—H) or hydroxyl (—OH); or a salt of said compound.

In a second aspect, the invention relates to a composition comprising said compound of the general formula I) in an amount of at least 0.25 mg/g, preferably of at least 0.5 mg/g, 1.0 mg/g or 1.5 mg/g, of the total composition.

Further aspects of the present invention relate to a use of said compound for enhancing the taste and/or saltiness of a food product.

A still further aspect of the present invention is a method for enhancing the taste and/or saltiness of a culinary food product, comprising the step of adding said compound or the composition comprising said compound to a food product.

The inventors surprisingly found that some sugar conjugates of glutamyl-serine and glutamyl-threonine dipeptides have a much stronger taste enhancing effect than their corresponding aglycones. In fact, these sugar conjugates enhance the saltiness and umami taste perception at much lower threshold levels than their corresponding aglycones. They also enhance the persistency of those tastes in the mouth and also reduce overall perceived bitterness of the products. The sugar conjugate molecules are typically generated in-situ during thermal processing of food raw materials by condensation of glucose with the corresponding dipeptides of glutamate with serine and threonine. The corresponding aglycones, i.e. the glutamyl-serine and glutamyl-threonine have been identified and described for example by S. Arai et al. in Agr. Biol. Chem. 37(1), 151-156 (1973), A. H. A. van den Oord and P. D. van Wassenaar in Z Lebensm Unters Forsch A (1997) 205: 125-130, and in EP2253227A1.

However, the taste properties of these dipeptides differ from the ones of their corresponding sugar conjugates. Evidence thereof is provided in the Example section below. Therefore, the molecules described in the present invention are more potent taste enhancers than the known corresponding dipeptides. They allow further reducing the amounts and uses of for example mono-sodium glutamate (MSG), of ribonucleotides such as IMP and GMP, and of regular kitchen salt in culinary food products and applications, without compromising flavor richness, deliciousness and salt perception of said products.

They also allow generating savory food concentrates which have much less or no MSG, ribonucleotides and/or salt, and which still provide a strong and typical delicious, umami and salt tasting effect if applied to a food product. It even allows generating such savory food concentrates which are much stronger and more concentrated in providing a salty taste to a food product upon application.

### BRIEF DESCRIPTION OF THE DRAWINGS

FIG. 1: Sensory evaluation of chicken soup spiked with 2 g/L GluAmadori-GluThr (dark columns) in comparison to un-spiked soup (grey columns). Sensory scores of the taste/flavor attributes are shown on a scale from 0 to 8. *) indicates statistic significant differences. The attributes are as follows: A) saltiness; B) bitterness; C) sweetness; D) boiled chicken; E) meaty; F) vegetables; G) umami; and H) overall flavor persistency.

### DETAILED DESCRIPTION OF THE INVENTION

The present invention pertains to a compound of the general formula I), wherein R1 is hydrogen (—H) or hydroxyl (—OH); or a salt of said compound.

Preferably, the compounds of the present invention are 1-deoxy-D-fructosyl-N-glutamyl-serine or 1-deoxy-D-fructosyl-N-glutamyl-threonine.

A second aspect of the invention relates to a composition comprising said compound of the general formula I) in an amount of at least 0.25 mg/g, at least 0.50 mg/g, at least 0.75 mg/g, at least 1.0 mg/g, at least 1.5 mg/g, at least 1.7 mg/g, at least 2 mg/g, at least 2.5 mg/g, at least 3 mg/g, at least 3.5 mg/g, or at least 5 mg/g of the total composition.

In one embodiment of the present invention, the composition is in the form of an extract from a plant, fungus and/or meat material. Preferably, the composition is in the form of an extract, for example from plant, fungus and/or meat material, where the compound of the present invention has been enriched. An advantage thereby is that the composition is of natural origin and does not contain any chemically synthesized compounds.

In another embodiment, the composition of the present invention is the result of a flavor reaction. The term “flavor reaction” refers herein to a chemical reaction occurring between at least one reducing sugar and at least one amino acid, peptide or protein. Typically, this chemical reaction occurs during a heating process and is typically also referred to as Maillard reaction. In one example, the flavor reaction is a Maillard reaction.

In a preferred embodiment, the composition of the present invention is food grade. Under “food grade” the inventors mean that the composition is suitable for human consumption, for example directly, in concentrated form, and/or when used diluted in a food product.

For example, the composition of the present invention is selected from the group consisting of a culinary seasoning product, a cooking aid, a sauce or soup concentrate, a dry or wet pet-food product.

Further aspects of the present invention relate to a use of said compound for enhancing the taste of a food product. Such a food product may be a ready-to-eat food product. It may also be a flavor concentrate used for seasoning a still further other food product. Advantageously, the compound of the present invention may be used for being added to a seasoning, a cooking aid or a food concentrate product. Thereby the strength of providing e.g. an umami or a salty taste to a still further food product is improved in such a seasoning, cooking aid or food concentrate product.

Particularly, the present invention relates to the use of the compounds for enhancing the umami and/or salt taste of a food product. More particularly, the invention relates to the use of the compounds of the present invention for enhancing the saltiness of a food product. Particularly, this use would allow to either increase the perceived saltiness of a food product without actually increasing the salt or sodium level of said food product, or to decrease the amount of salt or sodium used in a food product with maintaining the actual perceived saltiness of said product. Advantageously thereby the amount of salt and sodium consumed by consumers with such a product today could be significantly reduced.

Further aspects of the present invention also relate to a use of a composition comprising said compound in an amount of at least 0.25 mg/g, at least 0.50 mg/g, at least 0.75 mg/g, at least 1.0 mg/g, at least 1.5 mg/g, at least 1.7 mg/g, at least 2 mg/g, at least 2.5 mg/g, at least 3 mg/g, at least 3.5 mg/g, or at least 5 mg/g of the total composition, for enhancing the taste and/or saltiness of a food product. Advantageously, such a food product may be a ready-to-eat food product.

A still further aspect of the present invention is a method for enhancing the umami taste and/or saltiness of a culinary food product, comprising the step of adding said compound or the composition comprising said compound to a food product. The food product can be a ready-to-eat food product or a flavor concentrate.

Those skilled in the art will understand that they can freely combine all features of the present invention disclosed herein. In particular, features described for the products of the present invention may be combined with the uses and method of the present invention, and vice versa. Further, features described for different embodiments of the present invention may be combined. Further advantages and features of the present invention are apparent from the figures and examples.

### Example 1: Synthesis or Preparation of 1-deoxy-D-fructosyl-N-glutamyl-threonine

### Step-1: Synthesis of benzyl (4S)-5-(((2S)-1-(benzyloxy)-3-hydroxy-1-oxobutan-2-yl)amino)-4-((tert-butoxycarbonyl)amino) 5-oxopentanoate 3

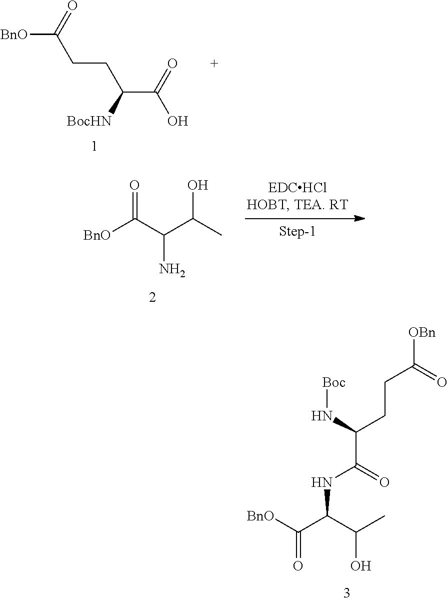 [SMILES: CC(C)(C)OC(=O)N[C@@H](CCC(=O)OCc1ccccc1)C(=O)O ; CC(O)C(N)C(=O)OCc1ccccc1 ; CC(O)[C@H](NC(=O)[C@H](CCC(=O)OCc1ccccc1)NC(=O)OC(C)(C)C)C(=O)OCc1ccccc1]

Dibenzyl (tert-butoxycarbonyl)-L-glutamate 1 (15.0 g, 44.510 mmol, 1.0 eq, supplied from Combi blocks) was dissolved in 600 mL dichloromethane. Benzyl-2-amino-3-hydroxybutanoate 2 (21.81 g, 89.020 mmol, 2.0 eq, TCI), 1-ethyl-3-(3-dimethylaminopropyl)carbodiimide hydrochloride (EDC.HCl) (10.20 g, 53.412 mmol, 1.5 eq.), hydroxybenzotriazole (HOBT) (3.00 g, 22.255 mmol, 0.5 eq.) and triethylamine (TEA) (13.48 g, 133.530 mmol, 3.0 eq.) were then added at room temperature. The reaction was stirred at room temperature for 6 hours. After completion, the reaction mass was diluted with 300 mL dichloromethane and washed with 150 mL saturated bicarbonate solution. Organic layer was dried over Na₂SO₄ and concentrated under reduced pressure to give a crude compound 3. The crude product was purified by column chromatography using neutral silica gel of 60-120 mesh size (0-50% ethyl acetate was used as gradient in hexane for elution) to finally lead to 15.0 g pure compound 3 (63.82% yield).

### Step-2: Synthesis of benzyl (4S)-4-amino-5-(((2S)-1-(benzyloxy)-3-hydroxy-1-oxobutan-2-yl)amino)-5-oxopentanoate 4

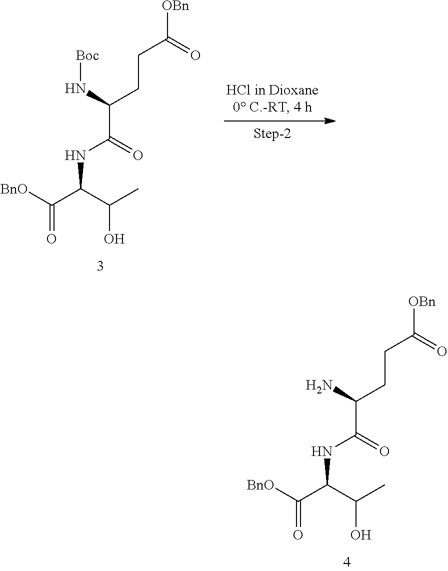 [SMILES: CC(O)[C@H](NC(=O)[C@H](CCC(=O)OCc1ccccc1)NC(=O)OC(C)(C)C)C(=O)OCc1ccccc1 ; CC(O)[C@H](NC(=O)[C@@H](N)CCC(=O)OCc1ccccc1)C(=O)OCc1ccccc1]

Benzyl (4S)-5-(((2S)-1-(benzyloxy)-3-hydroxy-1-oxobutan-2-yl)amino)-4-((tert-butoxycarbonyl)amino)-5-oxopentanoate 3 (15.0 g, 28.409 mmol, 1.0 eq.) was dissolved in 300 mL dichloromethane and HCl in 1,4-dioxane (4 M) was slowly added at 0° C. The resulting mixture was stirred at room temperature for 4 hours. The reaction mass was then concentrated to give 12.0 g pure final compound 4 (98.76% yield).

### Step-3: Synthesis of benzyl (4S)-5-(((2S)-1-(benzyloxy)-3-hydroxy-1-oxobutan-2-yl)amino)-5-oxo-4-(((2,3,4,5-tetrahydroxytetrahydro-2H-pyran-2-yl)methyl)amino)pentanoate 5

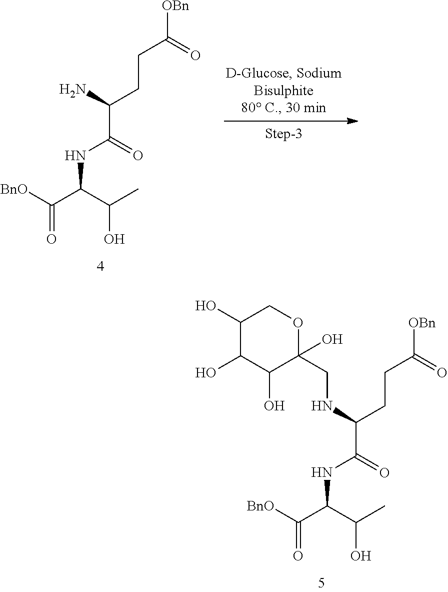 [SMILES: CC(O)[C@H](NC(=O)[C@@H](N)CCC(=O)OCc1ccccc1)C(=O)OCc1ccccc1 ; CC(O)[C@H](NC(=O)[C@H](CCC(=O)OCc1ccccc1)NCC1(O)OCC(O)C(O)C1O)C(=O)OCc1ccccc1]

D-Glucose (14.13 g, 78.504 mmol, 2.8 eq, SDfine Chemicals) and sodium bisulfite (0.816 g, 7.850 mmol, 0.28 eq.) were added in a mixture of 80 mL methanol and 20 mL glycerol. The reaction mixture was refluxed for 30 min at 80° C. followed by the addition of benzyl (4S)-4-amino-5-(((2S)-1-(benzyloxy)-3-hydroxy-1-oxobutan-2-yl)amino)-5-oxopentanoate 4 (12.0 g, 28.037 mmol, 1.0 eq.) and 7 mL acetic acid. The reaction mass was heated at 100° C. for further 5 hours. The reaction mass was then concentrated under reduced pressure to give a final crude compound 5 which was purified by column chromatography using neutral silica gel of 60-120 mesh size (0-7% methanol was used as gradient in dichloromethane for elution) to finally lead to 12.0 g pure compound 5 (81.63% yield).

### Step-4: Synthesis of (4S)-5-(((1S)-1-carboxy-2-hydroxypropyl)amino)-5-oxo-4-(((2,3,4,5-tetrahydroxy tetrahydro-2H-pyran-2-yl)methyl)amino)pentanoic acid

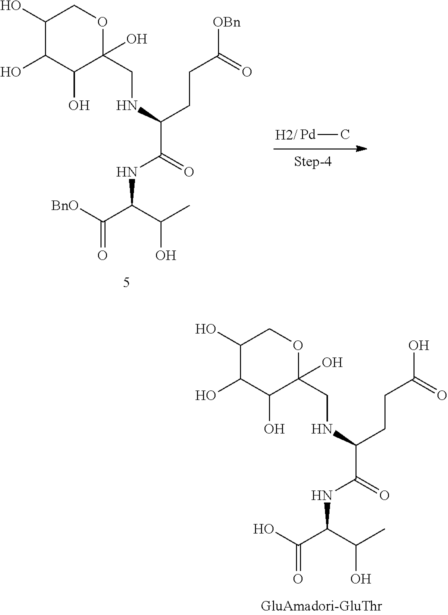

(4S)-5-(((1S)-1-carboxy-2-hydroxypropyl)amino)-5-oxo-4-(((2,3,4,5-tetrahydroxytetrahydro-2H-pyran-2-yl)methyl)amino)pentanoic acid 5 (13.0 g, 22.033 mmol, 1.0 eq.) was dissolved in 500 mL methanol and 10% Pd on Carbon (50% moisture) was slowly added. The resulting suspension was stirred at room temperature for 6 hours under H₂ gas atmosphere. The reaction mass was then filtered through Celite, washed with water and concentrated under reduced pressure to give a 7.0 g crude final compound. The crude product was finally purified by preparative HPLC to give 4.5 g pure final compound. (49.83% yield)

¹H NMR (360.13 MHz) and ¹³C NMR (90.56 MHz) spectra were recorded on a Bruker DPX-360 spectrometer equipped with a broadband multinuclear z-gradient probehead. The chemical shifts (in ppm) were expressed with respect to an internal reference (TMS or TSP). Multiplicities are reported as follows: s=singlet, d=doublet, t=triplet, q=quatruplet, m=multiplet, bs=broad singlet.

1H NMR (D2O) 1.057-1.073 (d, 3H), 2.139-2.154 (d, 2H), 2.525-2.537 (d, 2H), 3.194 (s, 2H), 3.372-3.402 (m, 1H), 3.500-3.518 (m, 1H), 3.602-3.669 (m, 1H), 3.701-3.759 (m, 1H), 3.854-3.860 (m, 1H), 3.930-3.977 (m, 1H), 4.0243-4.273 (m, 1H), 4.332 (s, 1H).

LC-MS analysis was carried out using X-Bridge C18 column (250*4.6 mm) at 202 nm. Column flow was 0.5 mL/min and solvents used were 20 mM ammonium acetate in HPLC grade water (A) and MeOH (B). Elution was as follows: from 0 to 11 min, % B was increased from 10% to 30%, then from 11 to 13 min, % B was increased from 30 to 90%, then stayed at 90% B to 15 min and finally decreased to 10% to 20 min: 411 (M+H.)

### Example 2: Synthesis or preparation of 1-deoxy-D-fructosyl-N-glutamyl-serine

1-deoxy-D-fructosyl-N-glutamyl-serine can be prepared analogous as 1-deoxy-D-fructosyl-N-glutamyl-threonine and as explicitly described in Example 1. For example the Benzyl-2-amino-3-hydroxybutanoate 2 in Step 1 could be replaced by Benzyl-2-amino-3-hydroxypropanoate and the rest of the chemical synthesis then adapted as necessary and obvious to a skilled person in the art.

### Example 3: Sensory Evaluation of the Compounds in Water

The compounds glutamyl-threonine and 1-deoxy-D-fructosyl-N-glutamyl-threonine were each dissolved and diluted in water in a final concentration of 2 g/L. The solutions were then evaluated by 12 panelists, which were previously screened and selected for their sensory abilities. The results of the sensory evaluation can be summarized as follows: the aqueous solution with the glutamyl dipeptides are slightly umami and salty as reported in the literature. However, the aqueous solution with the 1-deoxy-D-fructosyl-N-glutamyl-threonine was perceived as much more salty than the reference samples.

### Example 4: Sensory Evaluation of the Compounds in a Chicken Soup Base

Sample Preparation:

Chicken soups were prepared by dissolving 6 g chicken base powder (detailed recipe shown in Table 1) and 1 g monosodium glutamate in 500 mL hot water. 1-Deoxy-D-fructosyl-N-glutamyl-threonine was then added separately at 2 g/L final concentrations.

TABLE 1
Composition of the chicken base powder

| Ingredient | Quantity (wt %) |
|---|---|
| Chicken Meat powder | 30 |
| Starch | 1.52 |
| Flavors | 2.58 |
| Celery powder | 0.50 |
| Garlic powder | 0.90 |
| Chicken fat | 8.00 |
| Maltodextrine | 56.50 |
| Total | 100 |

Sensory Protocol:

The sensory evaluation was carried out by 12 panelists, previously screened for their sensory abilities. The panelists assessed a maximum of 6 samples per session. They had Vittel water and crackers as mouth cleansers. In all the cases, the panelists were instructed to evaluate the samples for the following attributes: overall flavor persistency, umami, meaty, grilled/popcorn-like, bread-like, boiled chicken, sweet, bitter, and salty. The samples were coded with random 3-digit numbers according to a balanced presentation design, heated at approximately 65° C. and then presented in 40 mL brown plastic containers and under red light to minimize appearance bias (the serving was approximately 25 mL per sample).

Statistical Analysis of the Results:

The following statistical tests were used to analyze the sensory raw data: First, the Analysis of Variance (ANOVA) with two factors, products (fixed factor) and subject (random factor) was computed in order to determine if there was any difference among samples. The limit of significance (alpha risk) was set at 5%. Then, the Least Significant Difference (LSD) multiple paired comparison test was performed when a significant difference was detected with the ANOVA, in order to determine which pairs of samples were significantly different. The limit of significance (alpha risk) was set at 5%.

Results of the Sensory Evaluation

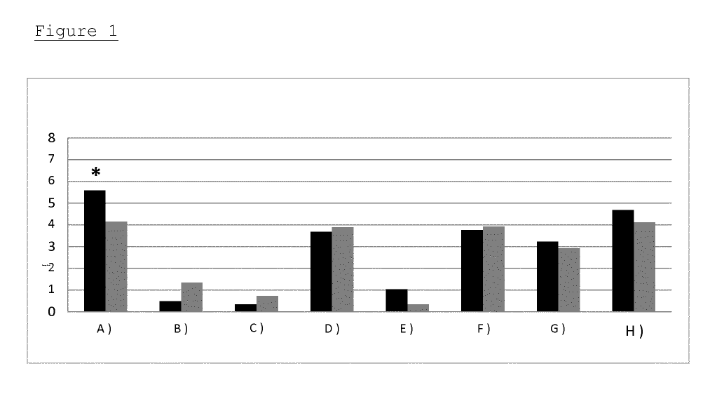

When comparing the reference samples with the sample comprising the 1-deoxy-D-fructosyl-N-glutamyl-threonine compound (chicken soup with 2 g/L of sugar conjugate of GluThr), saltiness was significantly increased (FIG. 1). There was also a clear trend that umami and meaty taste was improved in those samples. At the same time, bitterness was decreased.

### Example 5: Comparison Between the Soup Bases Containing the Sugar Conjugate 1-deoxy-D-fructosyl-N-glutamyl-threonine Compound and the Mixture of Glucose and glutamyl-threonine

A first soup was prepared by adding 2 g/L (4.57 mmol/L) 1-deoxy-D-fructosyl-N-glutamyl-threonine in the soup base described above under Example 4. A second soup was prepared by adding the corresponding same molar concentrations of glucose and glutamyl-threonine to the same soup base. The resulting soups were then evaluated by 6 panelists following the same procedure than described above under Example 4.

Obvious differences were found between the two sample soups as follows: When tasted with nose-clips, the soup comprising the added 1-deoxy-D-fructosyl-N-glutamyl-threonine was identified as significantly more salty and umami than the corresponding reference soup with the added glucose and glutamyl-threonine. And when tasted without nose-clips, the soup comprising the added 1-deoxy-D-fructosyl-N-glutamyl-threonine was identified as having clearly more enhanced savory flavors than the corresponding reference soup.

### Example 6: Seasoning Compositions

Tomato soups can be prepared by dissolving 6 g tomato base powder as can be obtained in the commerce in 500 mL hot water. 1-deoxy-D-fructosyl-N-glutamyl-threonine or alternatively 1-deoxy-D-fructosyl-N-glutamyl-serine can be added at a concentration of 0.5 g/L or 2.5 g/L to the soups in order to improve their taste and flavor profile. The soups will then have a more pronounced umami as well as being perceived as more salty than the corresponding reference soups without the addition of those compounds. For example, a similar tomato soup can now be prepared which has the same saltiness as the reference tomato soup but comprising less sodium chloride.

1. Compound of the general formula I) 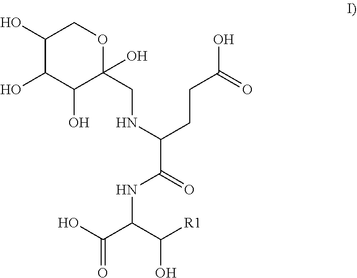
wherein R1 is hydrogen (—H) or hydroxyl (—OH); or a salt of the compound.

2. The compound according to claim 1, which is selected from the group consisting of 1-deoxy-D-fructosyl-N-glutamyl-serine and 1-deoxy-D-fructosyl-N-glutamyl-threonine.

3. A composition comprising the compound of the general formula I) 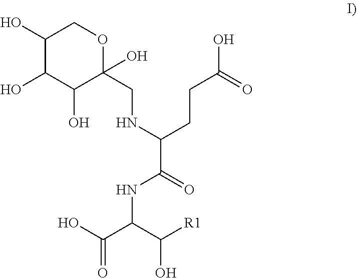
wherein R1 is hydrogen (—H) or hydroxyl (—OH); or a salt of the compound in an amount of at least 0.25 mg/g.

4. The composition according to claim 3, wherein the composition is food grade.

5. The composition according to claim 4, wherein the composition is selected from the group consisting of a culinary seasoning product, a cooking aid, a sauce or soup concentrate, a dry and a wet pet-food product.

6-8. (canceled)

9. Method for enhancing the saltiness of a culinary food product, comprising the step of adding the compound of the general formula I) 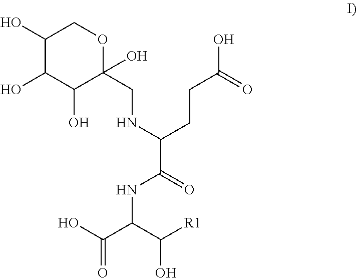
wherein R1 is hydrogen (—H) or hydroxyl (—OH); or a salt of the compound to a food product.

10. (canceled)
